(19) United States
(12) Patent Application Publication
(10) Pub. No.: US 2010/0018865 A1
(43) Pub. Date: Jan. 28, 2010

(54) ELECTROLYTE SOLUTION AND METHOD FOR ELECTROLYTIC CO-DEPOSITION OF THIN FILM CALCIUM PHOSPHATE AND DRUG COMPOSITES

(75) Inventors: Dean-Mo Liu, Richmond (CA); Mao-jung Maurice Lien, Mapla Ridge (CA); Doug Smith, Vancouver (CA); Manus Tsui, Richmond (CA); Arc Rajtar, Port Moody (CA)

Correspondence Address: RISSMAN HENDRICKS & OLIVERIO, LLP, 100 Cambridge Street, Suite 2101, BOSTON, MA, 02114, US

(73) Assignee: MIV Therapeutics Inc., Vancouver (CA)

(21) Appl. No.: 12/298,413

(22) PCT Filed: Apr. 26, 2007
(86) PCT No.: PCT/CA07/00707

§ 371 Date: Jul. 16, 2009

Related U.S. Application Data
(60) Provisional application No. 60/795,174, filed on Apr. 27, 2006.

(51) Int. Cl. C25D 5/00; A61K 47/00

(52) U.S. Cl. 205/147; 514/769

(57) ABSTRACT

Disclosed herein are electrolyte solutions and methods for electrolytic co-deposition of calcium phosphate and drug composites. The electrolyte solution may be formed by mixing solutions comprising calcium and phosphate precursors together to form an electrolyte solution. The electrolyte solution can have a water content less than 30 weight percent. The electrolyte solution may comprise a water-soluble non-aqueous solvent. A therapeutic agent, such as water-insoluble drug, is also present in the solution. The electrolyte solution thus formed may be used to co-deposit a calcium phosphate coating and the therapeutic agent on a substrate. One method includes the steps of immersing the substrate in the electrolyte solution and applying an electrical potential to the substrate to thereby cause (i) the calcium and phosphate precursors to electrochemically react with hydroxyl groups on the surface of the substrate and deposit the calcium phosphate coating thereon; and (ii) the therapeutic agent to electrophoretically migrate to the substrate and become co-deposited thereon together with the calcium phosphate coating. The method thus provides a convenient and easily controllable means for depositing thin film calcium phosphate and drug composites on substrates such as implantable medical devices.

### FIELD OF THE INVENTION

This application relates to an electrolyte solution and method for electrolytic co-deposition of calcium phosphate and drug composites.

### BACKGROUND OF THE INVENTION

Methods for the electrochemical deposition of calcium phosphate coatings such as hydroxyapatite are well-known in the prior art. For example, U.S. Pat. No. 5,759,376 and WO9607438, Teller et al., entitled “Method for the electrodeposition of hydroxyapatite layers,” describes the use of an electrolyte containing calcium phosphate and calcium hydrogen phosphate and a pulsed direct current of suitable frequency. The hydroxyapatite may be coated on metal or ceramic substrates. Once coated, such substrates are biocompatible and may be used in vivo as medical implants. For example, in one particular application, thin film cathodic electrodeposition may be used to coat implantable coronary stent surfaces.

U.S. Pat. No. 6,974,532, Legeros et al., teaches the use of a metastable calcium phosphate aqueous electrolyte solution to form a favorable adherent calcium phosphate coating on titanium-based biomedical devices via a modulated electrochemical deposition method, with a deposition temperature ranging from room temperature to 95° C. and pH 4 to pH 12, under pulse deposition current. However, no drug incorporation in the coating is described.

Pickford et al. in WO03039609 uses a two (or more) stage process involving a pre-treatment of the substrate surface followed by electrochemical deposition (preferably electrophoretical deposition) of hydroxyapatite coating on the pre-treated surface. The pre-treated surface is able to provide acceptable bond strength for the coating. The electrochemically-deposited hydroxyapatite coating is grown so as to provide reservoirs within the pores for pharmaceutically active compounds for slow drug delivery. However, no co-deposition of drug(s) is described.

Other prior art references also disclose the electrochemical deposition of calcium phosphate or hydroxyapatite coatings, either without drugs or later incorporation of drugs for therapeutic purposes: e.g. Lu et al., “Calcium phosphate crystal growth under controlled atmosphere in electrochemical deposition,” Journal of Crystal Growth, 284, pp. 506-516, 2005; Lin et al., “Adherent octacalciumphosphate coating on titanium alloy using modulated electrochemical deposition,” Journal of Biomed. Materials Research, 66A, 819-828, 2003; Kumar et al., “Electrodeposition of brushit coatings and their transformation to hydroxyapatite in aqueous solutions,” Journal of Biomed. Materials Research., 45, 302-310, 1999; Peng et al., “Thin calcium phosphate coatings on titanium by electrochemical deposition in modified simulated body fluid,” Journal of Biomed. Materials Research., 76A, 347-355, 2006; Ban et al., “Effect of temperature on electrochemical deposition of calcium phosphate coatings in a simulated body fluid,” Biomaterials, 16, 977-981, 1995; Cheng et al., “Electrochemically assisted co-precipitation of protein with calcium phosphate coatings on titanium alloy,” Biomaterials, 25, 5395-5403, 2004, where an all-water solution has to be used in this art, since the protein, i.e., bovine serum albumin, is water soluble; Huang et al. “A study of the process and kinetics of electrochemical deposition and the hydrothermal synthesis of hydroxyapatite coating,” Journal of Materials Science: Materials in Medicine, 11, 667-673, 2000; Magso et al., “Electrodeposition of hydroxyapatite coatings in basic conditions,” Biomaterials, 21, 1755-1761, 2000; and Hu et al, “Electrochemical deposition of hydroxyapatite with vinyl acetate on titanium implants,” Journal of Biomed. Materials Research, 65A, 24-29, 2003.

Since calcium phosphate coatings are naturally porous, they can be effectively employed as a scaffold for carrying organic materials, such as biopolymers, proteins or drugs. Impregnation of such organic materials in the porous voids within the coating may be achieved by various means, including co-deposition and post-deposition impregnation. In general, all of the prior art references referred to above employ water as a major diluting medium for therapeutically active water-soluble agents such as proteins and polymers. The prior art does not teach the co-deposition of a calcium phosphate coating and water-insoluble therapeutically active agent(s) (i.e. where water is not the major diluting medium for the therapeutically active agents). Although non-aqueous solvents have been employed for electrochemical application of metal coatings, such solvents are not typically used for electrochemical deposition or co-deposition of calcium phosphate coatings. In the case of co-deposition of calcium phosphate coatings and organic materials, the solution containing the organic material is principally water-based. There are several disadvantages to this conventional approach. First, if a high current is applied to trigger the electrochemical reactions, this may result in the formation of hydrogen gas bubbles at the cathodic substrate surface. The gas bubbles cause undesirable voids on the substrate surface, thus diminishing the bonding strength and uniformity of the coating. Second, the use of principally aqueous solutions may prevent the co-deposition of some organic materials, such as highly water-insoluble drugs. Third, the use of principally aqueous solutions may inhibit the electrophoretic migration of some drugs, especially when those drugs precipitate or are not able to be electrically charged in the presence of water (such as by protonization or ionization).

The need has therefore arisen for improved solutions and methods for electrolytic co-deposition of calcium phosphate and drug composites using water soluble non-aqueous solvent(s).

### SUMMARY OF THE INVENTION

In accordance with one embodiment of the invention, a method of forming an electrolyte solution is described. The method includes the steps of preparing a first solution comprising a calcium precursor; preparing a second solution comprising a phosphate precursor; mixing the first and second solutions to form a third solution comprising the calcium and phosphate precursors, wherein the third solution comprises a non-aqueous water-soluble solvent. The method further comprises adding a water-insoluble therapeutic agent to at least one of the first, second and third solutions. In one embodiment, the method comprises adding a water-insoluble therapeutic agent to the third solution. In one embodiment, the water content of the third solution is less than 30 weight percent.

Another embodiment of the invention relates to an electrolyte solution comprising a non-aqueous solvent; a water-insoluble therapeutic agent dissolved in the non-aqueous solvent; a calcium precursor; and a phosphate precursor, wherein the water content of the electrolyte solution is less than 30 weight percent.

Another embodiment of the invention relates to a method of co-depositing a calcium phosphate coating and a therapeutic agent on a substrate using any of the electrolyte solutions described herein. The method includes the steps of immersing the substrate in the electrolyte solution and applying an electric potential to the substrate to thereby cause (i) the calcium and phosphate precursors to electrochemically react and deposit the calcium phosphate coating on the substrate; and (ii) the therapeutic agent to electrophoretically migrate to the substrate (e.g., after being protonized or ionized in the electrolyte solution), and become co-deposited thereon together with the calcium phosphate coating.

### BRIEF DESCRIPTION OF THE DRAWINGS

Various embodiments of the invention will be understood from the following description, the appended claims and the accompanying drawings, which should not be construed as restricting the spirit or scope of the invention in any way.

FIG. 1 is a flowchart illustrating a multi-step procedure for synthesis of an electrolyte solution in accordance with the invention;

FIG. 2 is a schematic view showing electrically coupled cathodic and anodic electrodes for electrolytic co-deposition of calcium phosphate and drug composites; and

FIG. 3 is a schematic view showing putative formation of a drug-solvent-water molecule cluster in the electrolyte solution of the invention.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

One embodiment relates to an electrolyte solution useful for electrolytic co-deposition of calcium phosphate and drug composites. As described below, the co-deposition is achieved by a combination of electrochemical and electrophoretic processes.

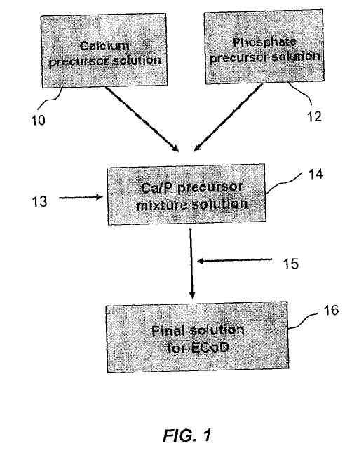

One general procedure for formulating the electrolyte solution is illustrated in FIG. 1. The first step in the procedure is the formation of a calcium precursor solution 10 and a phosphate precursor solution 12. As used in this patent application, “calcium precursor” means a calcium containing compound which may be used as a precursor to the formation of a calcium phosphate compound and “phosphate precursor” means a phosphate containing compound which may be used as a precursor to the formation of a calcium phosphate compound. Examples of calcium phosphate compounds include hydroxyapatite (Ca₁₀(Po4)₆(OH)₂) and tricalcium phosphate. Examples of calcium precursors include calcium salts such as calcium nitrate, calcium chloride, calcium lactate and calcium gluconate. Examples of phosphate precursors include phosphoric acid, phosphorus pentoxide and phosphate salts such as sodium phosphate, potassium phosphate and ammonium hydrogen phosphate.

As shown in FIG. 1, calcium precursor solution 10 is formed by dissolving a calcium precursor in an aqueous solvent (e.g. a calcium salt in water). As discussed below, a comparatively small amount of aqueous solvent is important to enable disassociation of metal salts to form ions. Phosphate precursor solution 12 is formed by dissolving a phosphate precursor in an aqueous or non-aqueous solvent. For example, phosphoric acid or phosphorus pentoxide may be dissolved directly in a non-aqueous water-soluble solvent such as methanol, ethanol, propanol, ethylene glycol, propylene glycol, butylene glycol, tetrahydrofuran (THF), N,N-dimethylacetamide (DMA), N,N-dimethylformamide (DMF) DMSO (dimethyl sulfoxide), N,N-diethylnicotinamide (DENA) or a mixture thereof. Alternatively, a phosphate salt, such as sodium phosphate and ammonium hydrogen phosphate, could be dissolved in a small amount of aqueous solvent (e.g. water) to form the phosphate precursor solution. In one embodiment of the invention the phosphate precursor solution 12 may comprise both aqueous and non-aqueous solvents.

As shown in FIG. 1, the calcium precursor solution 10 and phosphate precursor solution 12 are then mixed together. FIG. 1 also shows a step 13 of adding a therapeutic agent, such as a water-insoluble drug, to the mixture to form a therapeutic agent solution 14. Solution 14 may then be diluted with a water-soluble non-aqueous solvent (which may be the same or different from the solvents referred to above). In one embodiment, solution 14 is diluted with the water-soluble non-aqueous solvent until the water content of the solution is less than about 30 weight percent. The pH of solution 14 may also be adjusted to a value within the range of about 3 to 7, or a range of about 2 to 5, such as by adding potassium hydroxide or sodium hydroxide to the mixture, to form the final electrolyte solution 16 (FIG. 1). In some embodiments, the water content of solution 16 may be below 20 weight percent or below 10 weight percent. Electrolyte solution 10 is formulated in such embodiments such that solution 10 has a water content sufficient to completely dissolve the calcium precursor (and the phosphate precursor if it is water soluble) and to protonize the therapeutic agent upon the application of an electrical potential to the solution as described below. However, the water content of solution 10 should be maintained less than a threshold amount that would otherwise cause precipitation of the water-insoluble therapeutic agent. Thus, in certain embodiments, solution 10 comprises a combination of miscible aqueous and non-aqueous solvents wherein both the calcium and phosphate precursors and the therapeutic agent are completely dissolved in solution 10 (i.e. solution 10 is preferably clear with no visible solute precipitation).

In other possible embodiments of the invention, the therapeutic agent, or combination of agents, may be substantially or completely dissolved in solution 10 and/or 12 prior to mixing. In still other embodiments, the non-aqueous solvent or solvent mixture may be substantially rather than completely water-soluble. As will be apparent to a person skilled in the art, many variations are possible without departing from the invention. In various embodiments solution 10 is suitable for achieving co-deposition of calcium phosphate and drug composites according to the electrochemical and electrophoretic method described herein.

In one embodiment of the invention the molar ratio of the calcium to phosphate in solution 16 may range from about 1.0 to 1.70. The concentration of the calcium and phosphate constituents may be less than 1 weight percent in this example.

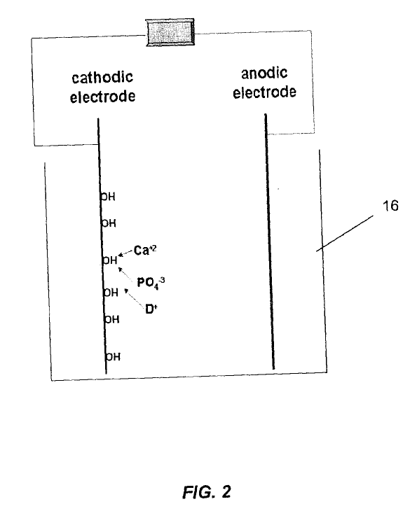

Electrolyte solution 16 may be used for co-deposition of calcium phosphate and drug composites on a electrically conductive substrate as shown schematically in FIG. 2. For example, the substrate may be a medical device, such as a stent, e.g., a metallic stent formed by a metal or metals including stainless steel, Co—Cr, Ti, Ti6Al4V and TiNi. The substrate may also be formed from other materials, or mixtures of materials, such as polymers, ceramics or carbon. As shown in FIG. 2, the co-deposition method is achieved by a combination of electrochemical and electrophoretic processes. The substrate may be a cathodic electrode which is immersed in electrolyte solution 16. When an electrical potential is applied to the substrate, the small amount of water in solution 16 enables the development of hydroxyl groups on the substrate surface. The calcium and phosphate precursors present in electrolyte solution 16 form ionic Ca and P species which chemically react with the hydroxyl groups at the interface between the cathodic electrode substrate (FIG. 2) and precipitate on the substrate. Simultaneously, the drug molecules present in electrolyte solution 16 acquire a positive electrical charge (D+) and are electrophoretically driven toward the cathodic substrate. The drug molecules may be deposited into intracrystal or intercrystal pores or voids in the growing calcium phosphate layer. The co-deposition process may be carried out at between ambient temperatures and a temperature of about 80° C.

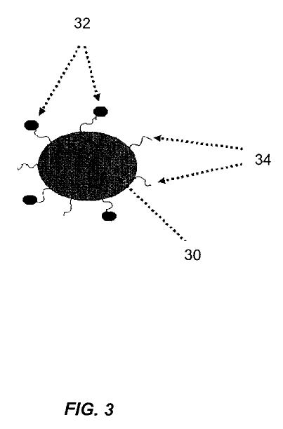

FIG. 3 illustrates the possible physical clusters of drug 30, solvent 34 and water molecules 32 in electrolyte solution 16. Unlike conventional electrolyte solutions, the relatively small amount of water molecules present in solution 16 are predominantly in a bonded rather than a free form. For example, the non-aqueous water-soluble solvent present in solution 16, such as DMSO, may chemically interact with multiple protonized water molecules by hydrogen bonding and/or dipole/dipole interactions. This feature is described, for example in Kirchner et al., “The Secret of Dimethyl Sulfoxide-Water Mixtures. A Quantum Chemical Study of 1 DMSO-nWater Clusters,” J. Am Chem. Soc., 124 (21), 6206-6215, 2002, the disclosure of which is incorporated herein by reference. Without wishing to be bound by any theory, it is believed that the drug 30, solvent 34 and water molecules 32 may form a positively charged molecular cluster (FIG. 3) which is driven electrophoretically to the cathodic substrate under an applied voltage. That is, the drug molecule is electrically charged via hydration with the solvent and the water molecules.

The co-deposition of calcium phosphate and drug composites using an electrolyte solution 16 having a relatively low water content can have one or more advantages over conventional electrochemical deposition processes. For example, it is possible to employ a relatively high current in the process without the formation of undesirable voids at the cathodic substrate surface due to the formation of hydrogen gas bubbles. Accordingly, a high calcium phosphate deposition rate and bonding strength may be controllably achieved. Further, the use of an electrolyte solution 16 substantially comprised of a water-soluble non-aqueous solvent may permit the incorporation of water-insoluble compounds such as drugs or highly reactive chemicals which may be driven to the substrate by electrophoretic processes. As explained above, the water content of solution 16 need only be sufficient to enable dissolving of the calcium precursor as well as protonization of the therapeutic agent and formation of hydroxyl groups on the cathodic substrate upon the application of an applied voltage.

The invention can provide an effective means of co-depositing a thin film calcium phosphate coating and drug nanocomposite on an electrically conductive substrate such as a medical device. Depending upon the makeup of electrolyte solution 16 and process parameters, the drug concentration in the nanocomposite may range from about 0.1 to 60 weight percent. The use of a water-soluble non-aqueous solvent may improve the solubility and bioavailability of the drug. The process may be useful for the deposition water-insoluble drugs including anti-cancer, anti-HIV, anti-inflammatory and anti-proliferative drugs.

As will be apparent to those skilled in the art, the invention could be used to co-deposit different types of drugs, including combinations of both water-insoluble and water-soluble drugs.

As will be apparent to those skilled in the art in the light of the foregoing disclosure, many other alterations and modifications are possible in the practice of this invention without departing from the spirit or scope thereof.

1. A method of forming an electrolyte solution containing a water-insoluble therapeutic agent, said method comprising:
(a) preparing a first solution comprising a calcium precursor;
(b) preparing a second solution comprising a phosphate precursor;
(c) mixing said first and second solutions to form a third solution comprising said calcium and phosphate precursors, wherein said third solution comprises a non-aqueous water-soluble solvent; and
(d) adding said water-insoluble therapeutic agent to at least one of said first, second, and third solutions.

2. The method according to claim 1, further comprising adjusting the pH of said electrolyte solution to a pH within the range of about 3-7.

3. The method according to claim 1, wherein said calcium precursor is a calcium salt and wherein preparing said first solution comprises dissolving said calcium salt in water.

4. The method according to claim 3, wherein said calcium salt is selected from the group consisting of calcium nitrate, calcium chloride, calcium lactate and calcium gluconate.

5. The method according to claim 1, wherein said phosphate precursor is a phosphate salt and wherein preparing said second solution comprises dissolving said phosphate salt in water.

6. The method according to claim 1, wherein preparing said second solution comprises dissolving said phosphate precursor in said non-aqueous water-soluble solvent.

7. The method according to claim 1, wherein said non-aqueous water-soluble solvent is selected from the group consisting of methanol, ethanol, propanol, ethylene glycol, propylene glycol, butylene glycol, THF, DMA, DMSO, DMF and DENA.

8. The method according to claim 6, wherein said non-aqueous water-soluble solvent is selected from the group consisting of methanol, ethanol, propanol, ethylene glycol, propylene glycol, butylene glycol, THF, DMA, DMSO, DMF and DENA.

9. The method according to claim 5, wherein said phosphate salt is selected from the group consisting of ammonium hydrogen phosphate, potassium phosphate and sodium phosphate.

10. The method according to claim 6, wherein said phosphate precursor is selected from the group consisting of consisting of phosphoric acid and phosphorus pentoxide.

11. The method according to claim 1, wherein the water content of said electrolyte solution is less than 30 weight percent.

12. The method according to claim 1, wherein said therapeutic agent is a water-insoluble drug.

13. The method according to claim 1, wherein said solution has a water content sufficient to completely dissolve said calcium precursor and to protonize said therapeutic agent upon the application of an electrical potential to said solution, but wherein said water content is less than a threshold amount that would cause precipitation of said therapeutic agent in said solution.

14. The method according to claim 1, comprising diluting said electrolyte solution with said non-aqueous water-soluble solvent until said non-aqueous water-soluble solvent comprises more than 70 weight percent of said electrolyte solution.

15. The method according to claim 12 or 13, wherein said therapeutic agent is completely dissolved in said electrolyte solution.

16. The method according to claim 1, wherein said first and second calcium phosphate precursors are completely dissolved in said electrolyte solution.

17. A method of co-depositing a calcium phosphate coating and a therapeutic agent on a substrate comprising:
(a) providing an electrolyte solution as defined in any one of claims 1-16;
(b) immersing said substrate in said electrolyte solution;
(c) applying an electrical potential to said substrate to thereby cause:
(i) said calcium and phosphate precursors to electrochemically react and deposit said calcium phosphate coating on said substrate; and
(ii) said therapeutic agent to electrophoretically migrate to said substrate and become co-deposited thereon with said calcium phosphate coating.

18. The method according to claim 17, wherein said therapeutic agent is encapsulated within said coating.

19. The method according to claim 17, wherein said method occurs at a temperature less than 80° C.

20. The method according to claim 17, wherein said therapeutic agent is electrically charged by hydration of said electrolyte solution.

21. The method according to claim 17, wherein said substrate is an implantable medical device.

22. The method according to claim 21, wherein said medical device has an outer surface selected from the group consisting of metal, ceramic and polymer materials.

23. The method according to any one of claims 17-22, wherein said calcium phosphate coating is hydroxyapatite.

24. An electrolyte solution comprising:
(a) a non-aqueous solvent;
(b) a water insoluble therapeutic agent dissolved in said solvent;
(c) a calcium precursor; and
(d) a phosphate precursor,
wherein the water content of said electrolyte solution is less than 30 weight percent.

25. The solution according to claim 24, wherein any water molecules in said solution are chemically or physically bound to said non-aqueous first solvent.

26. The solution according to claim 24, wherein said non-aqueous solvent is water-soluble.

27. The solution according to claim 24, wherein the water content of said electrolyte solution is less than 20 weight percent.

28. The solution according to claim 24, wherein the pH of said solution is within the range of about 3-7.

29. The solution according to claim 26, wherein said non-aqueous water-soluble solvent is selected from the group consisting of methanol, ethanol, propanol, ethylene glycol, THF, DMA, DMSO, DMF and DENA.

30. The solution according to claim 24, wherein said calcium precursor is a calcium salt selected from the group consisting of calcium nitrate, calcium chloride, calcium lactate and calcium gluconate.

31. The solution according to claim 24, wherein said phosphate precursor is a phosphate salt selected from the group consisting of ammonium hydrogen phosphate and sodium phosphate.

32. The solution according to claim 24, wherein said phosphate precursor is selected from the group consisting of consisting of phosphoric acid and phosphorus pentoxide.

33. The solution according to claim 24, wherein said therapeutic agent is a water insoluble drug.

34. The solution according to claim 24, wherein said solution has a water content sufficient to completely dissolve said calcium precursor and to protonize said therapeutic agent upon the application of an electrical potential to said solution, but wherein said water content is less than a threshold amount that would cause precipitation of said therapeutic agent in said solution.

35. The solution according to claim 24, wherein said first and second precursors are completely dissolved in said solution.

36. The solution according to claim 25, wherein said solution comprises water molecules and wherein said therapeutic agent is electrically charged in the presence of said non-aqueous solvent in an acidic environment with the range of pH 3-7.

37. An electrolyte solution or method of making or using same comprising any new, useful and inventive feature, combination of features or sub-combination of features, described or clearly inferred herein.
